(19) United States
(12) Patent Application Publication
(10) Pub. No.: US 2009/0286799 A1
(43) Pub. Date: Nov. 19, 2009

(54) METHODS FOR THE TREATMENT OF BRAIN EDEMA

(76) Inventor: ZHI-GANG JIANG, Gaithersburg, MD (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 12/467,757

(22) Filed: May 18, 2009

Related U.S. Application Data
(60) Provisional application No. 61/114,444, filed on Nov. 13, 2008.
(60) Provisional application No. 61/053,668, filed on May 16, 2008.

(51) Int. Cl. A61K 31/4965; A61K 31/44; A61K 31/426; A61K 31/4164; A61P 7/10

(52) U.S. Cl. 514/252.1; 514/357; 514/365; 514/400

(57) ABSTRACT

The present invention is based on the discoveries that PAN-811 (1) reduces infarct volume, suppresses brain edema and decreases mortality associated with ischemia; (2) blocks veratridine-induced swelling and neuronal cell death; (3) chelates free calcium and inhibits MMP-9 activity; and (4) blocks calcium-induced neuronal cell death and suppresses glutamate-induced calcium influx into neuronal cells. More particularly, the present invention relates to methods for treating, ameliorating or preventing vasogenic and/or cytotoxic brain edema, by administering to a subject in need thereof certain thiosemicarbazone compounds or pharmaceutically acceptable salts thereof. An example of such a thiosemicarbazone is 3-aminopyridine-2-carboxaldehyde thiosemicarbazone (PAN-811).

### CROSS-REFERENCE TO RELATED APPLICATIONS

The present application claims the benefit of U.S. Provisional Application No. 61/114,444, filed Nov. 13, 2008, and of U.S. Provisional Application No. 61/053,668, filed May 16, 2008, which are incorporated by reference herein in their entireties.

### BACKGROUND OF THE INVENTION

Edema refers to the presence of excess interstitial fluid (or fluid in spaces between cells) in tissues. Thus, the presence of excess fluid or increases in interstitial fluid pressure that increase fluid volume can result in edema. Edema of the brain is characterized by an excess accumulation of water in the intracellular and/or extracellular spaces of the brain. Brain edema can cause dysfunction of the affected part of the brain, compression of brain tissue and brain herniation, potentially leading to brain damage and death.

Brain edema may be broken down into two categories, vasogenic edema and cytotoxic edema. Vasogenic brain edema is caused by mechanical or autodigestive disruption or functional breakdown of the endothelial cell layer of brain vessels (an essential structure of the blood-brain barrier (BBB)). Breakdown of this endothelial cell layer allows for uncontrolled ion and protein transfer from the intravascular to the extracellular (interstitial) brain compartments and water accumulation in the brain. Anatomically, this pathology increases the volume of the extracellular space (DeWitt D S, Prough D. J Neurotrauma. 2003; 20:795-825; Unterberg A W, Stover J, Kress B, Kiening K L. 2004; 129:1021-9). Cytotoxic brain edema is characterized by intracellular water accumulation in neurons, astrocytes, and microglia, irrespective of the integrity of the endothelial cell layer. Cytotoxic brain edema may be caused by an increase in cell membrane permeability for ions, ionic pump failure due to energy depletion, and cellular reabsorption of osmotically active solutes (Unterberg et al., 2004; Stiefel M F, Tomita Y, Marmarou A. J. Neurosurg. 2005; 103:707-14).

Clinically, hyperosmolar therapy agents (e.g. mannitol, glycerol, albumin and hypertonic saline), corticosteroids, indomethacin and barbiturates are commonly used for suppressing brain edema (Ayata C, Ropper A H. J Clin Neurosci. 2002; 9:113-124). However, the therapeutic outcome of these agents is not satisfactory either due to inconsistency between experimental models, lack of efficacy or side effects. The unsatisfactory outcome of current therapies is at least in part due to our limited knowledge about the sequence of pathological events causing edema, such as the mechanisms underlying BBB disruption (Montaner J, Rodriguez-Yanez M, Castellanos M, Alvarez-Sabin J, Castillo J. Seminars in Cerebrovascular Diseases and Stroke. 2005; 5:178-188). Therefore, there remains a need for alternative treatment options for brain edema and a need to further understand the mechanisms underlying BBB disruption, in order to provide a potent approach to treating brain edema and rescue the lives of patients.

N-heterocyclic carboxaldehyde thiosemicarbazones (HCTs) are useful as antineoplastic agents, acting as potent inhibitors of ribonucleotide reductase. Methods of treatment of tumors using such compounds, e.g., Triapine®, Vion Pharmaceuticals, Inc., are disclosed, inter alia, in U.S. Pat. Nos. 5,721,259 and 5,281,715 of Sartorelli et al. Moreover, carbocyclic and heterocyclic substituted thiosemicarbazones, such as those disclosed in U.S. Pat. No. 6,613,803, are useful for the treatment of neuronal damage and neurodegenerative diseases. These thiosemicarbazone compounds are described as exerting their therapeutic effects as sodium channel blockers. See U.S. Pat. No. 6,613,803. For example, PAN-811 (3-aminopyridine-2-carboxaldehyde thiosemicarbazone), a divalent metal chelator, has been shown to protect ischemic and/or oxidative stress-induced neuronal cell death via chelation of intracellular free calcium and scavenging of free radicals (Jiang Z G, Lu X C, Nelson Y, Yang X, Pan W, Chen R W, Lebowitz M S, Almassian B, Tortella F C, Brady R O, Ghanbari H A. Proc Natl Acad Sci USA. 2006; 103(5):1581-6; Jiang Z G, Lebowitz M S, Ghanbari H A. CNS Drug Reviews. 2006; 12:77-90).

### BRIEF SUMMARY OF THE INVENTION

The present invention provides a method for treating, ameliorating or preventing vasogenic and/or cytotoxic brain edema comprising administering to a subject in need of treatment a therapeutically effective amount of a N-heterocyclic carboxaldehyde thiosemicarbazone (HCT), e.g., PAN-811. The present invention is based on the discoveries that PAN-811 (1) reduces infarct volume, suppresses brain edema and decreases mortality associated with ischemia; (2) blocks veratridine-induced swelling and neuronal cell death; (3) chelates free calcium and inhibits MMP-9 activity; and (4) blocks calcium-induced neuronal cell death and suppresses glutamate-induced calcium influx into neuronal cells. More particularly, the present invention relates to methods of treating or reducing brain edema, including vasogenic and cytotoxic edema, by administering to a subject in need thereof certain thiosemicarbazone compounds. An example of such a thiosemicarbazone is 3-aminopyridine-2-carboxaldehyde thiosemicarbazone (PAN-811). Based on these discoveries, the invention relates generally to methods of treating or reducing brain edema by the administration of certain N-heterocyclic 2-carboxaldehyde thiosemicarbazones (HCTs) and pharmaceutically acceptable salts thereof. Such useful compounds are embraced by Formula I:

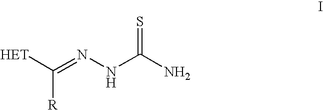

where HET is a 5 or 6 membered heteroaryl residue having 1 or 2 heteroatoms selected from N and S, and optionally substituted with an amino group; and R is H or C₁-C₄-alkyl.

In certain embodiments, the compound is selected from a compound of Formulas II-VI, infra.

In certain embodiments, PAN-811 (3-aminopyridine-2-carboxaldehyde thiosemicarbazone) is used to practice the methods of the present invention, which has the formula:

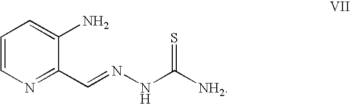

### BRIEF DESCRIPTION OF THE DRAWINGS

FIG. 1A shows the effects of PAN-811 treatment on infarct volume in rats with middle cerebral artery occlusion (MCAo).

FIG. 1B shows the effects of PAN-811 treatment on edema volume in rats with MCAo.

FIG. 1C shows the percent mortality of MCAo rats treated with PAN-811 or vehicle.

FIGS. 2A-2D show histological photographs of neurons pre-treated with PAN-811 or vehicle, prior to insult with veratridine, as well as the cellular area and lactate dehydrogenase (LDH) and 3-(4,5-dimethylthiazol-2-yl)-5-(3-carboxymethoxyphenyl)-2-(4-sulfophenyl)-2H-tetrazolium (MTS) analyses of these cells.

FIG. 3A shows lactate dehydrogenase (LDH) analysis of neurons pre-treated with PAN-811 or vehicle prior to insult with veratridine.

FIG. 3B shows the effects of PAN-811 (PAN) or MK-801 (MK) treatment on glutamate (glut)-induced neuronal cell death, as measured by fluorescence intensity.

FIG. 4 shows a schematic for the measurement of MMP-9 bioactivity by the MMP Assay Kit (AnaSpec Corp., San Jose, Calif.).

FIG. 5 shows experimental results for determining the optimal concentration of 5-FAM for the MMP Assay, reported as relative fluorescence units (RFU).

FIGS. 6A and 6B show the effects of PAN-811 (PAN) or vehicle (Veh) treatment on MMP-9 bioactivity in vitro, reported as relative fluorescence units (RFU).

### DETAILED DESCRIPTION OF THE INVENTION

### Definitions

Unless defined otherwise, all technical and scientific terms used herein have the same meaning as commonly understood by one of ordinary skill in the art to which this invention belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present invention, suitable methods and materials are described below. The materials, methods and examples are illustrative only and are not intended to be limiting. Other features and advantages of the invention will be apparent from the detailed description and from the claims.

In order to further define this invention, the following terms and definitions are provided.

It is to be noted that the term “a” or “an” entity, refers to one or more of that entity; for example, “a polypeptide,” is understood to represent one or more polypeptides. As such, the terms “a” (or “an”), “one or more,” and “at least one” can be used interchangeably herein.

As used herein, a “therapeutically effective amount” refers to an amount effective, at dosages and for periods of time necessary, to achieve a desired therapeutic result as described elsewhere herein. A therapeutic result may be, e.g., lessening of symptoms caused by brain edema, prolonged survival of a subject suffering from brain edema, and the like. A therapeutic result need not be a “cure”. As used herein, a “a subject in need of therapeutic and/or preventative immunity” refers to a subject which it is desirable to treat, i.e., to prevent, cure, retard, or reduce the severity of brain edema, and/or result in no worsening of brain edema over a specified period of time.

By “subject” or “individual” or “animal” or “patient” or “vertebrate” or “mammal,” is meant any subject, e.g., a mammalian subject, for whom therapy is desired. Mammalian subjects include, but are not limited to, humans; domestic animals; farm animals; zoo animals; sport animals; pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; ursids such as bears, food animals such as cows, pigs, and sheep; ungulates such as deer and giraffes; rodents such as mice, rats, hamsters and guinea pigs; and so on. In certain embodiments, the subject is a human subject.

As used herein, the terms “treat” and “treatment” refers to therapeutic treatment, including prophylactic or preventative measures, wherein the object is to prevent or slow down (lessen) an undesired physiological change or disorder, such as the progression of brain edema. Beneficial or desired clinical results include, but are not limited to, alleviation of the symptoms of brain edema, diminishment of the extent of brain edema, stabilized (i.e., not worsening) state of brain edema, delay or slowing of brain edema progression, amelioration or palliation of the brain edema, and remission (whether partial or total) of brain edema, whether detectable or undetectable. “Treatment” can also mean prolonging survival as compared to expected survival if not receiving treatment. Those in need of treatment include those already experiencing brain edema due to a certain disease, disorder, condition, or injury, or due to other therapeutic treatments, as well as those prone to brain edema due to a certain disease, disorder, condition, or injury, or due to other therapeutic treatments.

The present invention relates to methods of treating, ameliorating, reducing or preventing brain edema, including vasogenic and cytotoxic edema, by administering to a subject in need thereof certain thiosemicarbazone compounds or pharmaceutically acceptable salts thereof. The present invention also relates to methods of treating or reducing brain edema, including vasogenic and cytotoxic edema, by administering to a subject in need thereof a therapeutically effective amount of a compound of Formula I, or a pharmaceutically acceptable salt thereof:

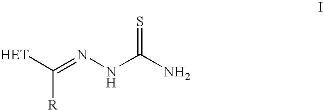

wherein HET is a 5 or 6 membered heteroaryl residue having 1 or 2 heteroatoms selected from N and S, and optionally substituted with an amino group; and R is H or C₁-C₄-alkyl.

In certain embodiments, the present invention relates to methods of treating or reducing brain edema, including vasogenic and cytotoxic edema, by administering to a subject in need thereof a therapeutically effective amount of a compound of Formula II, or a pharmaceutically acceptable salt thereof:

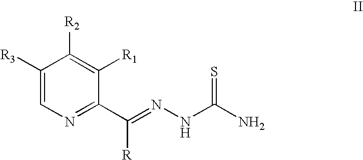

wherein R is H or C₁-C₄-alkyl; and R₁, R₂ and R₃ are independently selected from H and amino.

In certain embodiments, the present invention relates to methods of treating or reducing brain edema, including vasogenic and cytotoxic edema, by administering to a subject in need thereof a therapeutically effective amount of a compound of Formula III, or a pharmaceutically acceptable salt thereof:

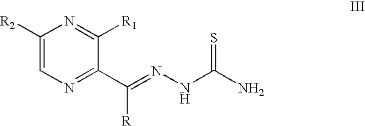

wherein R is H or C₁-C₄-alkyl; and R₁ and R₂ are independently selected from H and ammo.

In certain embodiments, the present invention relates to methods of treating or reducing brain edema, including vasogenic and cytotoxic edema, by administering to a subject in need thereof a therapeutically effective amount of a compound of Formula IV, or a pharmaceutically acceptable salt thereof:

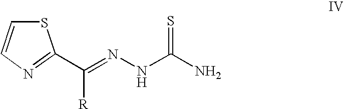

wherein R is H or C₁-C₄-alkyl.

In certain embodiments, the present invention relates to methods of treating or reducing brain edema, including vasogenic and cytotoxic edema, by administering to a subject in need thereof a therapeutically effective amount of a compound of Formula V, or a pharmaceutically acceptable salt thereof:

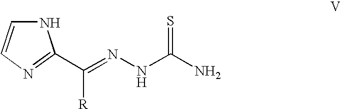

wherein R is H or C₁-C₄-alkyl.

In certain embodiments, the present invention relates to methods of treating or reducing brain edema, including vasogenic and cytotoxic edema, by administering to a subject in need thereof a therapeutically effective amount of a compound of Formula VI, or a pharmaceutically acceptable salt thereof:

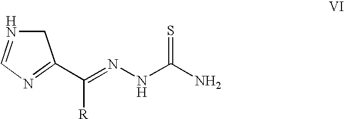

wherein R is H or C₁-C₄-alkyl.

In certain embodiments, the present invention relates to methods of treating or reducing brain edema, including vasogenic and cytotoxic edema, by administering to a subject in need thereof a therapeutically effective amount of a compound of Formula VII, or a pharmaceutically acceptable salt thereof:

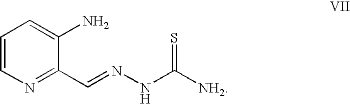

In certain embodiments, the present invention relates to thiosemicarbazones of a the following formulas, or a pharmaceutically acceptable salt thereof: (1) Formula II, where R is methyl, and R₁, R₂ and R₃ are H; (2) Formula III, where R is methyl and R₁ and R₂ are H; (3) Formula IV, where R is methyl; (4) Formula IV, where R is H; (5) Formula V, where R is H; and (6) Formula VI, where R is H.

Examples of thiosemicarbazones of the present invention are described in U.S. Patent Publication Nos. 20060160826, 20060194810 and 20070179147, which are hereby specifically incorporated by reference.

Certain of the compounds of the present invention may exist as E, Z-stereoisomers about the C═N double bond and the invention includes the mixture of isomers as well as the individual isomers that may be separated according to methods that are well known to those of ordinary skill in the art. Certain of the compounds of the present invention may exist as optical isomers and the invention includes both the racemic mixtures of such optical isomers as well as the individual enantiomers that may be separated according to methods that are well known to those of ordinary skill in the art.

Examples of pharmaceutically acceptable salts are inorganic and organic acid addition salts such as hydrochloride, hydrobromide, phosphate, sulphate, citrate, lactate, tartrate, maleate, fumarate, acetic acid, dichloroacetic acid and oxalate.

It is unexpected that thiosemicarbazones, including the compound, 3-aminopyridine-2-carboxaldehyde thiosemicarbazone, are effective in treating and reducing brain edema because, although iron chelation has been used to reduce ischemia/reperfusion-induced brain edema (Patt A, Horesh I R, Berger E M, Harken A H, Repine J E. J Pediatr Surg. 1990; 25(2):224-7), thiosemicarbazones have not been previously used for treating an edema condition.

Diseases, disorders, conditions or injuries associated with edema include, but are not limited to: infarction, for example, cerebral artery infarction or brain infarction; injury, for example, brain injury, head injury, spinal cord injury, traumatic brain injury, traumatic head injury or traumatic spinal cord injury; trauma, for example, head trauma, cerebral trauma or spinal cord trauma; cerebral venous thrombosis; intracerebral hemorrhage; ischemia, for example, brain ischemia, hemorrhagic ischemia or cerebral ischemia; acute disseminated encephalitis (ADEM); stroke, for example, ischemic stroke; tumors, for example, brain tumor or spinal cord tumor; brain infection; brain abscess; surgery; post-surgical manipulation; sepsis; hypertension; respiratory insufficiency; poisoning, for example, CO poisoning, tin poisoning, lead poisoning or arsenical poisoning; hyponatremia; acute nephropathy; hepatic encephalopathy; disequilibrium syndrome caused by hemodialysis; hyperglycemia; hypoglycemia; adrenal insufficiency; collagen diseases; blood-central nervous system barrier dysfunction; or optical diseases, for example, diabetic retinopathy.

Diseases, disorders, conditions, or injuries associated with vasogenic edema include, but are not limited to: brain ischemia, brain infarction, intracranial hemorrhage from neurosurgical operations, brain infections and abscesses, brain tumor and traumatic head injuries.

Diseases, disorders, conditions, or injuries associated with cytotoxic edema include, but are not limited to: intracerebral hemorrhage, cerebral contusion, cerebral infarction, brain tumor, stroke, drug-induced lung injury, drug-induced pulmonary disease, anthrax toxicity, hepatic encephalopathy, influenza encephalopathy, intracranial hypertension, hepatic failure, hepatic encephalopathy, nephrotic syndrome, diabetes, sarcoidosis, high altitude, and altitude sickness.

The means for synthesizing compounds useful in the methods of the invention are well known in the art. Such synthetic schemes are described in U.S. Pat. Nos. 5,281,715; 5,767,134; 4,447,427; 5,869,676; and 5,721,259, all of which are incorporated herein by reference in their entireties.

In certain embodiments, the invention is directed to using pharmaceutical compositions of the thiosemicarbazones in the methods of the invention. The pharmaceutical compositions comprise one or more of the compounds (or one of the compounds together with one or more different active ingredients) and a pharmaceutically acceptable carrier or diluent. As used herein “pharmaceutically acceptable carrier or diluent” includes any and all solvents, dispersion media, solid excipients (e.g., binders, lubricants, etc. typically used in solid oral dosage forms) coatings, antibacterial and antifungal agents, isotonic and absorption delaying agents, and the like that are physiologically compatible. The type of carrier can be selected based upon the intended route of administration. In certain embodiments, the carrier comprises 7% (v/v) polyethylene glycol (PEG) 300, 3% ethanol (EtOH), 28.6 mmol/L citric acid and 5.7 mmol/L L-ascorbic acid solution.

In certain embodiments, the carrier is suitable for intravenous, intracranial, intraperitoneal, subcutaneous, intramuscular, topical, transdermal or oral administration.

For example, pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. In all dosage forms, supplementary active compounds may be incorporated into the compositions as well.

In certain embodiments, administration is oral, and may be of an immediate or delayed release. Such oral pharmaceutical compositions of the present invention are manufactured by techniques typically used in the pharmaceutical industry. Generally, the active agent(s) is/are formulated into a tablet or capsule for oral administration, prepared using methods known in the art, for instance wet granulation and direct compression methods. The oral tablets are prepared using any suitable process known to the art. See, for example, Remington's Pharmaceutical Sciences, 18^{th} Edition, A. Gennaro, Ed., Mack Pub. Co. (Easton, Pa. 1990), Chapters 88-91, the entirety of which is hereby incorporated by reference. Typically, the active ingredient, i.e., one or more of the thiosemicarbazones, is mixed with pharmaceutically acceptable excipients (e.g., the binders, lubricants, etc.) and compressed into tablets. Such a dosage form may be prepared, for example, by a wet granulation technique or a direct compression method to form uniform granulates. Alternatively, the active ingredient(s) can be mixed with a previously prepared non-active granulate. The moist granulated mass is then dried and sized using a suitable screening device to provide a powder, which can then be filled into capsules or compressed into matrix tablets or caplets, as desired.

In certain embodiments, the tablets are prepared using a direct compression method. The direct compression method offers a number of potential advantages over a wet granulation method, particularly with respect to the relative ease of manufacture. In the direct compression method, at least one pharmaceutically active agent and the excipients or other ingredients are sieved through a stainless steel screen, such as a 40 mesh steel screen. The sieved materials are then charged to a suitable blender and blended for an appropriate time. The blend is then compressed into tablets on a rotary press using appropriate tooling.

In certain embodiments, the pharmaceutical composition is contained in a capsule containing beadlets or pellets. Methods for making such pellets are known in the art (see, Remington's, supra). The pellets are filled into capsules, for instance gelatin capsules, by conventional techniques.

Sterile injectable solutions can be prepared by incorporating a desired amount of the active compound in a pharmaceutically acceptable liquid vehicle and filter sterilized. Generally, dispersions may be prepared by incorporating the active compound into a sterile vehicle containing a basic dispersion medium. In the case of sterile powders for the preparation of sterile injectable solutions, exemplary methods of preparation are vacuum drying and freeze-drying, which will yield a powder of the active ingredient plus any additional desired ingredient from a previously sterile-filtered solution thereof.

In certain embodiments, the active agent(s) in the pharmaceutical composition (i.e., one or more of the thiosemicarbazones, e.g., PAN-811) is present in a therapeutically effective amount. By a “therapeutically effective amount” is meant an amount effective, at dosages and for periods of time necessary, to achieve the desired therapeutic result of positively influencing the course of a particular disease state. This terminology also contemplates and encompasses the therapeutic use of the compounds in a prophylactic manner, which may be of a lower dosage, and such an embodiment is included in the present invention. Of course, therapeutically effective amounts of the active agent(s) may vary according to factors such as the disease state, age, sex, and weight of the individual, and the ability of the agent to elicit a desired response in the individual. Dosage regimens may be adjusted to provide the optimum therapeutic response. A therapeutically effective amount is also one in which any toxic or detrimental effects of the agent are outweighed by the therapeutically beneficial effects.

The amount of active compound in the composition may vary according to factors such as the disease state, age, sex, and weight of the individual. Dosage regimens may be adjusted to provide the optimum therapeutic response. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals. It is contemplated that the dosage units of the present invention will contain the active agent(s) in amounts suitable for a dosage regimen of about the same as, or less than, those presently employed in antineoplastic treatment (e.g., Triapine®, Vion Pharmaceuticals, Inc.).

In certain embodiments, the dosage or amount of compound useful in the methods of the invention is typically about 0.1 mg/kg to about 10 mg/kg of the subject's body weight. In certain embodiments, the dosage or amount of compound is from about 0.5 mg/kg to about 5 mg/kg of the subject's body weight, or from about 0.5 mg/kg to about 2 mg/kg of the subject's body weight. In certain embodiments, the dosage or amount of compound is about 0.5 mg/kg, about 1 mg/kg, about 2 mg/kg, or about 5 mg/kg of the subject's body weight. In certain embodiments, the dosage or amount of compound is from about 5 mg/m²/day to about 160 mg/m²/day, or from about 96 mg/m²/day to about 160 mg/m²/day, or from about 2.6 mg/kg to about 4.3 mg/kg of the subject's body weight.

The compositions used in the methods of the present invention may be administered by any suitable method, e.g., parenterally, intracranial, intraventricularly, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional, intravenous, intraarterial, intraperitoneal, rectal, vaginal, and intracranial injection or infusion techniques.

In certain embodiments, the compounds useful in the methods of the invention are delivered as early as possible upon the onset of a disease, disorder, condition, or injury associated with edema, including, but not limited to, stroke or brain injury. In certain embodiments, the compounds useful in the methods of the invention may be delivered, for example, about 1 hour, about 2 hours, about 6 hours, about 12 hours or about 24 hours after the onset of a disease, disorder, condition, or injury associated with edema, including, but not limited to, stroke or brain injury. In certain embodiments, the compounds useful in the methods of the invention may be delivered, for example, no later than about 1 hour, about 2 hours, about 6 hours, about 12 hours, or about 24 hours after the onset of a stroke or brain injury. It is contemplated that the compounds useful in the methods of the invention may also be administered after 24 hours.

In certain embodiments, the thiosemicarbazones of the invention may be administered alone or in combination with other compounds, or with other compositions. A thiosemicarbazone on the invention, e.g., PAN-811, may be administered with other therapeutic agents, including, but not limited to, tissue plasminogen activator (t-PA), MMP inhibitors, T-477, NS-7, superoxide dismutase (SOD), and hyperosmotic agents (mannitol, glycerol, and hypertonic saline solutions). The dosage of t-PA administered to a subject is typically about 0.1 mg/kg to about 10 mg/kg of the subject's body weight by injection or by infusion.

Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

In certain embodiments, the pharmaceutical compositions of the invention may be administered to any subject in need of the beneficial effects of the compounds of the invention. While the invention is primarily directed to human use, other animals and mammals may be treated accordingly if so desired. Examples of mammals include humans, dogs, cats, mice, rats, rabbits sheep, cattle, horses and pigs.

This invention is further illustrated by the following examples, which are not intended to limit the present invention. The contents of all references, patents, and published patent applications cited throughout this application are specifically and entirely incorporated herein by reference.

### EXAMPLES

### Example 1

### PAN-811 Reduces Infarct Volume in Middle Cerebral Artery Occlusion (MCAo) Rats

The anti-edema properties of PAN-811 were demonstrated in a rat model as follows.

Ischemic stroke is caused by the occlusion of an artery to the brain and results in brain infarction and cerebral edema (Bounds J V, Wiebers D O, Whisnant J P, Okazaki H. Stroke. 1981; 12:474-477; Silver F L, Norris J W, Lewis A J, Hachinski V C. Stroke. 1984; 15:492-496; Moulin D E, Lo R, Chiang J, Barnett H J. Stroke. 1985; 16:282-284; Dávalos A, Toni D, Iweins F, Lesaffre E, Bastianello S, Castillo J. Stroke. 1999; 30:2631-2636). Model systems, such as Middle Cerebral Artery Occlusion (MCAo), have been developed whereby ischemic stroke is induced in an animal by manual occlusion of a cerebral artery to study ischemic stroke, brain infarction and cerebral edema.

### Materials

For Middle Cerebral Artery Occlusion (MCAo), male Sprague-Dawley rats (270 to 330 g) were anesthetized with gaseous isoflurane. With aseptic surgical techniques, a laser-Doppler probe was placed on the right temporal-lateral portion of the skull over a region of the cortex which is supplied by the middle cerebral artery to record cerebral blood flow (CBF). CBF was recorded before and after arterial occlusion. The bifurcation of the right external common carotid 8 artery (ECA) and internal common carotid artery (ICA) was surgically exposed. A 4-0 monofilament suture treated with a silicone coating to enlarge the tip to better achieve an arterial block was placed into the ECA stump, and advanced up the ICA approximately 20 mm until a drop in CBF of approximately 75% or greater was measured by laser Doppler flowmetry (PeriFlux PF5010, Perimed). The duration of the ischemia was 120 minutes.

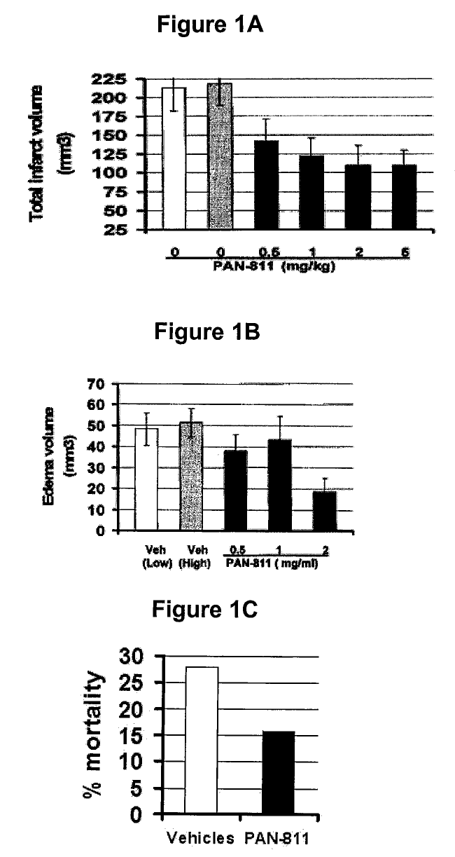

PAN-811 was originally dissolved to a concentration of 25.6 mmol/L in 7% (v/v) polyethylene glycol (PEG) 300, 3% ethanol (EtOH), 28.6 mmol/L citric acid and 5.7 mmol/L L-ascorbic acid solution. 25.6 mmol/L was the highest concentration of PAN-811 used for dosing. This solution was further diluted with normal saline to produce dosing solutions of 2.56 mmol/L, 5.12 mmol/L, and 10.2 mmol/L on the day of surgery. Vehicle dosing solutions were identical in components to the 5.12 mmol/L PAN-811 group (Vehicle Low), or the 10.2 mmol/L group (Vehicle High). These treatments were delivered intravenously route into MCAo rats at reperfusion time. Specifically, 1 mL/kg of each above preparation was injected into the penile vein at reperfusion (2 hrs after onset of occlusion) to achieve in vivo PAN-811 doses of 0, 0.5, 1, 2 and 5 mg/kg (black bars, FIG. 1). Low dose and high dose vehicle treatments were also performed as negative controls (open bars and grey bars, FIG. 1, respectively). Table 1 summarizes these treatments, doses and the number of animals (No.) tested (data presented in FIG. 1A, as described below).

TABLE 1
Summary of Treatments, Doses and Number of Animals Tested.

| Treatment | Dose | No. |
|---|---|---|
| Vehicle (Low) | 1 mg/kg | 13 |
| Vehicle (High) | 2 mg/kg | 14 |
| PAN-811 | 0.5 mg/kg | 11 |
| PAN-811 | 1 mg/kg | 10 |
| PAN-811 | 2 mg/kg | 11 |
| PAN-811 | 5 mg/kg | 10 |

After 22 hours of treatment, the animals were sacrificed by CO₂ asphyxiation, and their brains sectioned at 2 mm intervals. Brain sections were stained with 2,3,5-triphenyltetrazolium chloride and fixed in formalin. Brain areas were traced and measured using a computerized image analysis system (SimplePCI Version 4.0.0, Compix Inc) to determine infarct volume (in cubic millimeters, mm³), whereby unstained areas were defined as ischemic lesions of infarcted tissue.

### Results

A 2-hour MCAo resulted in total infarct volume of approximately 200-225 mm³ (FIG. 1A). The MCAo rats that received different dose levels of PAN-811 showed a dose-dependent reduction of infarct volume (FIG. 1A).

### Example 2

### PAN-811 Reduces Edema Volume in Middle Cerebral Artery Occlusion (MCAo) Rats

The effect of PAN-811 on edema volume in Middle Cerebral Artery Occlusion (MCAo) Rats was tested as follows.

### Results

Ischemic stroke often results in brain edema (Ayata et al., 2002). Therefore, the volumes for both ipsilateral and contralateral side hemispheres of MCAo rats were measured 22 hours later, and the edema volumes (hemisphere volume of the ipsilateral side hemisphere volume of contralateral one) were calculated, and expressed as mean±SEM (FIG. 1B). A 2-hour MCAo resulted in an increase of ipsilateral hemisphere volume by approximately 50 mm³. The MCAo rats that received different dose levels of PAN-811 showed a trend in reduction of edema volume. A 70% reduction in edema volume was achieved when MCAo rats were treated with 2 mg/kg PAN-811 when compared to the high vehicle group. However, 5 mg/kg PAN-811 treatment achieved only 31% reduction in edema volume by comparing with High vehicle group (data not shown).

### Example 3

### PAN-811 Reduces the Mortality of Middle Cerebral Artery Occlusion (MCAo) Rats

The effect of PAN-811 on mortality of Middle Cerebral Artery Occlusion (MCAo) rats was tested as follows.

### Results

Brain edema can result in death of MCAo rats. Therefore, the mortalities for vehicle groups and PAN-811 treated groups, as explained in Examples 1 and 2, were counted (FIG. 1C). Mortalities of general vehicle treated groups (open bar) and collective PAN-811 treated groups (black bar) were determined and premature death rates were expressed as percentage of total animal number in each condition. The premature mortality rate was 28% (11 of 39 animals) for the 2 vehicle-treated groups. In contrast, the premature mortality rate for all PAN-811 treated groups was 15.8% (9 of 57). Thus, the premature mortality in PAN-811 treated groups was reduced by 44%.

These results show that PAN-811 reduces the mortality of MCAo rats. Taken together, Examples 1-3 show PAN-811 efficiently suppresses infarct volume, brain edema and premature death of focal ischemic rats, indicating that PAN-811 has an excellent potential application in stroke therapy.

### Example 4

### PAN-811 Protects Veratridine-Induced Neuronal Swelling, Lactate Dehydrogenase (LDH) Release and Morphological Cell Death

The effect of PAN-811 on veratridine-induced neuronal swelling, lactate dehydrogenase (LDH) release and morphological cell death in neuronal cells was tested as follows.

Veratridine, a Na⁺ channel opener, results in both Na⁺ and Ca²⁺ influx, and a 1.8-2.0 fold increase in cell volume of cultured rat cortical neurons (Churchwell K B, Wright S H, Emma F, Rosenberg P A, Strange K. J Neurosci. 1996; 16:7447-7457). Veratridine-induced cell swelling results in cell membrane leakage, causing release of lactate dehydrogenase (LDH) and eventually cell death (Ramnath et al., 1992). As such, veratridine insult can be used in cell models to study Na⁺ and Ca²⁺ influx and cell death in neurons.

### Methods

Mixed cultures of neurons were isolated from the cortex and striatum of embryonic day 17 male Sprague-Dawley rats and seeded at 5×10⁴ cells per well in 96-well plates. To obtain highly enriched neurons (˜95%), neurons were cultured in Neurobasal medium containing B27 supplement with antioxidants (AO). Prior to treatment, AO concentration in the culture medium was reduced to 25% by replacing the medium twice with culture medium lacking AO. The neurons were pre-treated overnight with 1.25, 2.5 or 5 μM PAN-811 (PAN) or with the corresponding 1:20,000, 1:10,000, 1:5,000 dilutions of vehicle (Veh) (7:3 PEG:EtOH diluted with balanced salt solution (BSS)) as a carrier control.

The neurons were then insulted without or with 10 μM veratridine (Ver) for a 48-hour period. At the end of the experiment, the cultures were photographed under an inverted phase contrast microscope at a magnification of 300×. Nine of the largest neurons were selected and measured for cellular area by using ImageJ program (the National Institutes of Health) and statistically analyzed with a one-factor ANOVA by using VASSARSTATS (http://faculty.vassar.edu/lowry/VassarStats.html). Cell membrane integrity was evaluated by measuring lactate dehydrogenase (LDH) and mitochondrial function was evaluated using the MTS assay (Jiang Z G, Lu X C, Nelson Y, Yang X, Pan W, Chen R W, Lebowitz M S, Almassian B, Tortella F C, Brady R O, Ghanbari H A. Proc Natl Acad Sci USA. 2006; 103(5):1581-6; Jiang Z G, Lebowitz M S, Ghanbari H A. CNS Drug Reviews. 2006; 12:77-90).

In the MTS assay, mitochondrial function is evaluated by the ability of active mitochondrial reductase enzyme to reduce yellow-colored MTS (3-(4,5-dimethylthiazol-2-yl)-5-(3-carboxymethoxyphenyl)-2-(4-sulfophenyl)-2H-tetrazolium), in the presence of phenazine methosulfate (PMS), into the water-soluble, purple-colored formazan product that has an absorbance maximum at 490-500 nm in phosphate-buffered saline.

### Results

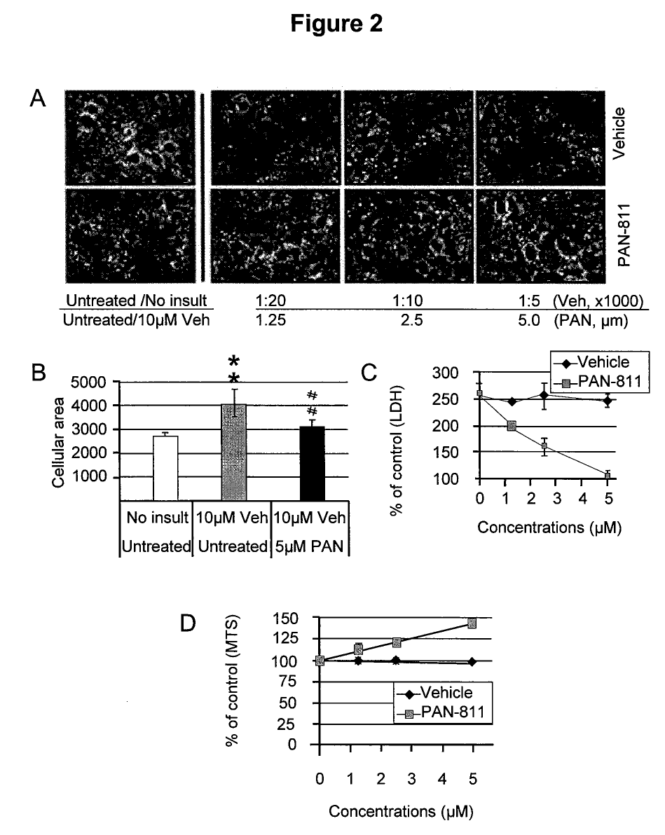

Morphological neuronal cell death was observed in veratridine-insulted groups which were pre-treated without or with different dilutions of vehicle (FIG. 2A). Veratridine insult caused a significant increase in neuronal cell size compared to untreated controls that were not insulted (P<0.01) (FIG. 2B). Treatment with 5 μM PAN-811 prior to veratridine insult resulted in a significant decrease in neuronal cell size compared to untreated controls that were insulted (P<0.01) (FIG. 2B).

Veratridine insult resulted in a 1.5-fold increase in neuronal LDH release (FIG. 2C, -♦-). PAN-811 pre-treatment dose-dependently reduced veratridine-induced LDH efflux and achieved full reversal of veratridine-induced LDH efflux at concentration of 5 μM (FIG. 2C, -▪-).

Veratridine insult did not affect MTS readings (FIG. 2D, -♦-). However, pre-treatment with PAN-811 dose-dependently increased MTS readings, indicating that PAN-811 treatment improved mitochondrial function over control level (FIG. 2D, -▪-).

These results show that PAN-811 treatment protects veratridine-induced neuronal swelling, LDH release and morphological cell death.

### Example 5

### PAN-811 Blocks Calcium-Induced Neuronal Cell Death and Suppresses Glutamate-Induced Calcium Influx

The effect of PAN-811 on calcium-induced neuronal cell death and glutamate-induced calcium influx in neuronal cells was tested as follows.

Glutamate is the brain's primary excitatory neurotransmitter. Binding of glutamate to glutamate receptors results in an influx of calcium in neurons. MK-801, also known as dizocilpine, is a non-competitive antagonist of the N-methyl-d-aspartate (NMDA) glutamate receptor, and inhibits glutamate-mediated calcium influx in neurons.

### Methods

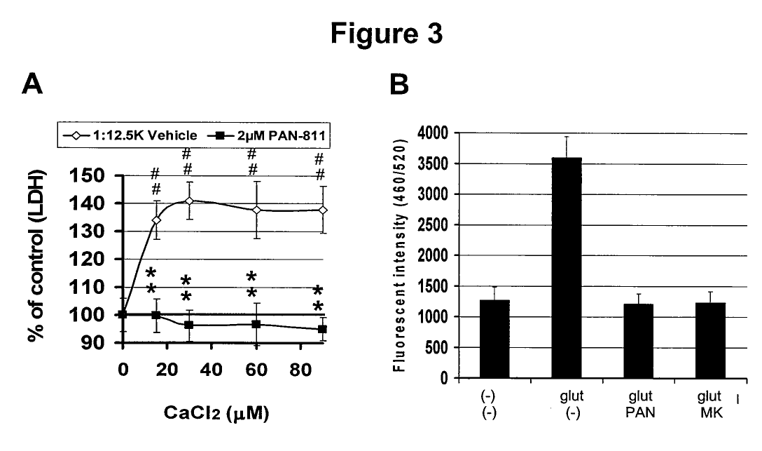

To understand the underlying mechanism for reducing cytotoxic brain edema, rat cortical and striatal neurons isolated as described in Example 4 were cultured in medium containing 12.5% antioxidants (by replacing culture medium 3 times with one containing no antioxidants in B27 supplement) for 30 days and co-treated with different concentrations of calcium chloride (CaCl₂), 1:12,500-fold diluted vehicle (FIG. 3A, -⋄-), or 2 μM PAN-811 (FIG. 3A, -▪-) for another 10 days. Cell membrane damage was evaluated by examination of lactate dehydrogenase (LDH) concentrations in culture medium as described in Example 4.

In addition, neurons were cultured for 16 days, treated with 20 μM glutamate (glut) alone or together with 5 μM PAN-811 (PAN) or MK-801 (MK) for 30 min, and further co-incubated for 30 min with 10 μM Calcium Green-1, a fluorescent calcium ion indicator dye. The fluorescence intensity of Calcium Green-1 dye was examined under an inverted fluorescence microscope and quantified with FluoStar plate reader at excitation 460 m and emission 520 nm. Data were expressed as means (n=6) and standard deviation.

### Results

Treatment with 2 μM PAN-811 reversed calcium-induced LDH elevation (FIG. 3A) and also suppressed glutamate-induced calcium influx (FIG. 3B).

### Example 6

### PAN-811 Inhibits Bioactivity of Calcium/Zinc-Dependent Matrix Metalloproteinase-9 (MMP-9)

The effect of PAN-811 on the bioactivity of calcium/zinc-dependent matrix metalloproteinase-9 (MMP-9) was tested as follows.

### MMP Assay Kit Protocol for Measuring MMP-9 Bioactivity

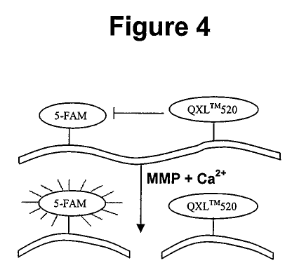

FIG. 4 shows a schematic representation of the MMP Assay Kit protocol for measuring MMP-9 bioactivity (AnaSpec Corp., San Jose, Calif.). The principle behind this analysis is as follows. A 5-FAM/QXL™520 fluorescence resonance energy transfer (FRET) peptide is used as a substrate for MMP. The fluorescence of 5-FAM is quenched by QXL™520 in intact FRET peptide. However, upon cleavage of FRET into two separate fragments by MMP, in the presence of Ca²⁺, the fluorescence of 5-FAM is recovered, and can be monitored at excitation/emission wavelengths of 490 mm/520 nm with a fluorometer. Thus, an inhibitor of MMP-9 could suppress the cleavage of FRET by MMP-9 and decrease the fluorescence intensity of 5-FAM.

### Methods

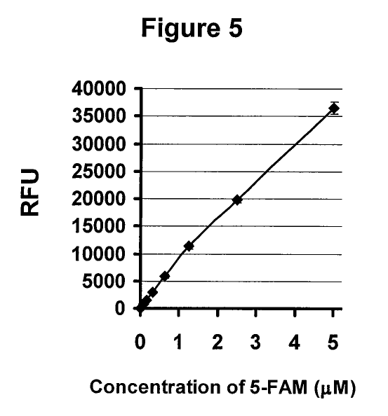

To determine the optimal concentration of 5-FAM for the MMP Assay Kit, 1 mM of 5-FAM was diluted to final concentrations of 2.5, 1.25, 0.625, 0.315, 0.156, and 0.078 μM and added to experimental plate wells as described in detail below. Fluorescence intensity of 5-FAM was determined by measurement at excitation/emission wavelengths of 490 nm/520 nm with a fluorometer and reported as relative fluorescence units (RFU) (FIG. 5).

To test the effect of PAN-811 on the bioactivity of MMP-9, black 96-well plates (Cat #3603, Costar®), Corning Inc.) were pre-warmed at 37° C. 4-aminophenylmercuric acetate (APMA) was diluted 20-fold with assay buffer (both in MMP assay kit) to a final concentration of 50 mM. Recombinant human pro-MMP-9 (10 μg/ml, Cat #72009, AnaSpec, San Jose) was pre-incubated with 1 mM APMA at 37° C. for 2 hours to activate this enzyme. An aliquot of 46 μl MMP substrate (5-FAM/QXL™520 FRET peptide, 100-fold diluted with assay buffer) was added in each well and the plates were preincubated at 37° C. for 15 min.

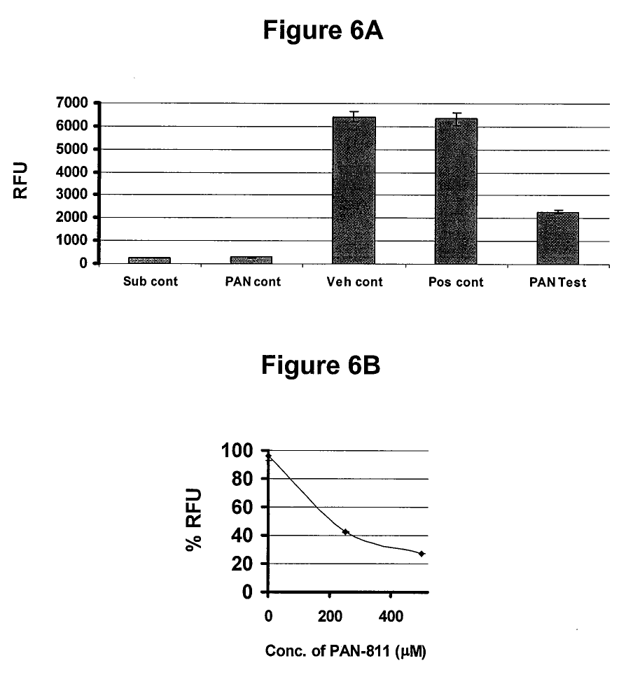

After pre-incubation, one of following treatments was performed in each well: (1) 4 μl of assay buffer was supplemented as a substrate control lacking activated pro-MMP-9 (Sub cont; FIG. 6A); (2) 2 μl of assay buffer and 2 μl of activated pro-MMP-9 were supplemented as a positive control (Pos cont; FIG. 6A); (3) 2 μl of activated pro-MMP-9 and 2 μl of vehicle (7:3 PEG:EtOH for dissolving PAN-811) were supplemented as a vehicle control (Veh cont; FIG. 6A); (4) 2 μl of assay buffer and 2 μl of 25 mM PAN-811 were supplemented as a test compound control lacking activated pro-MMP-9, to account for possible autofluorescence of the test compound (PAN cont; FIG. 6A); and (5) 2 μl of 5 μg/ml of activated pro-MMP-9 and 2 μl of 6.25, 12.5 or 25 mM PAN-811 were supplemented as the experimental test (PAN Test; FIG. 6A). Triple replicates were performed for each treatment. Treated wells were incubated at room temperature for 30 min and the fluorescence intensity of 5-FAM was determined for each sample by measurement at excitation/emission wavelengths of 485 nm/520 nm with a fluorometer and reported in RFU (FIGS. 6A and 6B). The final concentration of PAN-811 was 1 mM (200 ng/ml) and the calcium concentration (from storage buffer for pro-MMP-9) was 0.4 mM.

### Results

Vehicle and positive control samples supplemented with pro-MMP-9 exhibited a 23-fold increase in fluorescence intensity (RFU) compared to control samples lacking pro-MMP-9 (FIG. 6A). Incubation with PAN-811 suppressed pro-MMP-9-catalyzed increases in fluorescence intensity by 64% (FIG. 6B).

These results show that PAN-811 inhibits the bioactivity of MMP-9.

Taken together, these Examples indicate that: (a) PAN-811 significantly reduces ischemia-induced (cytotoxic) brain edema and consequent high mortality in a rat model; and (b) PAN-811 blocks veratridine-induced neuronal cell death and decreases the bioactivity of MMP-9, both established model systems for vasogenic edema.

What is claimed is:

1. A method of treating, ameliorating, reducing, or preventing brain edema, comprising administering to a subject in need thereof a therapeutically effective amount of a compound of Formula I, or a pharmaceutically acceptable salt thereof: 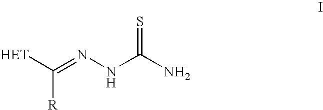
wherein HET is a 5 or 6 membered heteroaryl residue having 1 or 2 heteroatoms selected from N and S, and optionally substituted with an amino group; and R is H or C₁-C₄-alkyl.

2. The method of claim 1, wherein the compound or salt thereof is of Formula II: 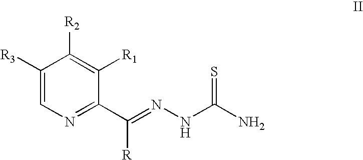
wherein R is H or C₁-C₄-alkyl; and R₁, R₂ and R₃ are independently selected from H and amino.

3. The method of claim 1, wherein the compound or salt thereof is of Formula III: 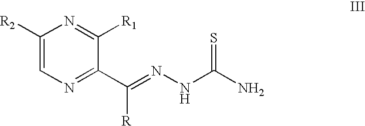
wherein R is H or C₁-C₄-alkyl; and R₁ and R₂ are independently selected from H and amino.

4. The method of claim 1, wherein the compound or salt thereof is of Formula IV: 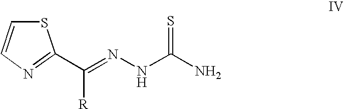
wherein R is H or C₁-C₄-alkyl.

5. The method of claim 1, wherein the compound or salt thereof is of Formula V: 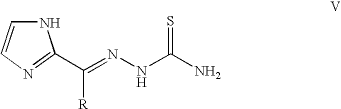
wherein R is H or C₁-C₄-alkyl.

6. The method of claim 1, wherein the compound or salt thereof is of Formula VI: 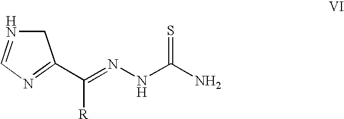
wherein R is H or C₁-C₄-alkyl.

7. The method of claim 1, wherein the compound or salt thereof is of Formula VII:

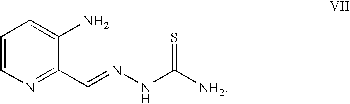

8. The method of claim 2, wherein R is methyl and R₁, R₂ and R₃ are H.

9. The method of claim 3, wherein R is methyl and R₁ and R₂ are H.

10. The method of claim 4, wherein R is methyl.

11. The method of claim 5, wherein R is H.

12. The method of claim 6, wherein R is H.

13. The method of claim 1, wherein the pharmaceutically acceptable salt is selected from the group consisting of hydrochloride, hydrobromide, phosphate, sulphate, citrate, lactate, tartrate, maleate, fumarate, acetic acid, dichloroacetic acid, and oxalate.

14. The method of claim 1, wherein the subject is a mammal.

15. The method of claim 14, wherein the mammal is a human.

16. The method of claim 1, wherein the compound or salt thereof is administered in a pharmaceutical composition comprising one or more pharmaceutically acceptable carriers or diluents.

17. The method of claim 16, wherein the carrier comprises 7% (v/v) polyethylene glycol (PEG) 300, 3% ethanol (EtOH), 28.6 mmol/L citric acid and 5.7 mmol/L L-ascorbic acid solution.

18. The method of claim 1, wherein the compound or salt thereof is administered as early as possible upon the onset of the disease, disorder, condition, or injury associated with edema.

19. The method of claim 1, wherein the disease, disorder, condition, or injury associated with edema is stroke.

20. The method of claim 1, wherein the compound or salt thereof is administered in combination with an additional therapeutic agent.

21. The method of claim 20, wherein the additional therapeutic agent is tissue plasminogen activator.

22. The method of claim 20, wherein the additional therapeutic agent is a hyperosmotic agent.

23. The method of claim 20, wherein the additional therapeutic agent is a diuretic or corticosteroid.

24. The method of claim 1, wherein the compound or salt thereof is administered intravenously.

25. The method of claim 1, wherein the compound or salt thereof is administered intracranially.

26. The method of claim 1, wherein the compound or salt thereof is administered at a dosage of from about 0.1 mg/kg to about 10 mg/kg of the subject's body weight.

27. The method of claim 26, wherein the compound or salt thereof is administered at a dosage of from about 0.5 mg/kg to about 5 mg/kg of the subject's body weight.

28. The method of claim 27, wherein the compound or salt thereof is administered at a dosage of from about 0.5 mg/kg to about 2 mg/kg of the subject's body weight.

29. The method of claim 1, wherein the compound or salt thereof blocks sodium channels.

30. The method of claim 1, wherein the compound or salt thereof chelates free calcium.

31. The method of claim 1, wherein the edema is cytotoxic edema.

32. The method of claim 1, wherein the edema is vasogenic edema.

33. The method of claim 1, wherein the edema is associated with infarction.

34. The method of claim 33, wherein the infarction is cerebral artery infarction or brain infarction.

35. The method of claim 1, wherein the edema is associated with injury.

36. The method of claim 35, wherein the injury is brain injury, head injury, spinal cord injury, traumatic brain injury, traumatic head injury, or traumatic spinal cord injury.

37. The method of claim 1, wherein the edema is associated with trauma.

38. The method of claim 37, wherein the trauma is head trauma, cerebral trauma, or spinal cord trauma.

39. The method of claim 1, wherein the edema is associated with cerebral venous thrombosis.

40. The method of claim 1, wherein the edema is associated with intracerebral hemorrhage.

41. The method of claim 1, wherein the edema is associated with ischemia.

42. The method of claim 41, wherein the ischemia is brain ischemia, hemorrhagic ischemia, or cerebral ischemia.

43. The method of claim 1, wherein the edema is associated with acute disseminated encephalitis (ADEM).

44. The method of claim 1, wherein the edema is associated with stroke.

45. The method of claim 44, wherein the stroke is ischemic stroke.

46. The method of claim 1, wherein the edema is associated with tumor.

47. The method of claim 46, wherein the tumor is a brain tumor or spinal cord tumor.

48. The method of claim 1, wherein the edema is associated with poisoning.

49. The method of claim 48, wherein the poisoning is carbon monoxide (CO) poisoning, tin poisoning, lead poisoning, or arsenic poisoning.

50. The method of claim 1, wherein the edema is associated with optical disease.

51. The method of claim 50, wherein the optical disease is diabetic retinopathy.

52. The method of claim 1, wherein the edema is associated with inflammation.

53. The method of claim 1, wherein the edema is associated with a disease, disorder, condition or injury selected from the group consisting of brain infection, brain abscess, surgery, post-surgical manipulation, sepsis, hypertension, respiratory insufficiency, hyponatremia, acute nephropathy, hepatic encephalopathy, disequilibrium syndrome caused by hemodialysis, hyperglycemia, hypoglycemia, adrenal insufficiency, collagen diseases, blood-central nervous system barrier dysfunction, and altitude sickness.

54. A method of treating, ameliorating, reducing, or preventing a disruption of, or an increase in permeability of, the blood-brain barrier comprising administering to a subject in need thereof a therapeutically effective amount of a compound of Formula I, or a pharmaceutically acceptable salt thereof: 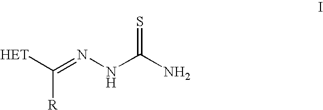
wherein HET is a 5 or 6 membered heteroaryl residue having 1 or 2 heteroatoms selected from N and S, and optionally substituted with an amino group; and R is H or C₁-C₄-alkyl.
